(19) United States
(12) Patent Application Publication
(10) Pub. No.: US 2017/0348349 A1
(43) Pub. Date: Dec. 7, 2017

(54) Modulation of Amyloid Beta Toxicity and Processing By Xenon Gas

(71) Applicant: NOBLIS THERAPEUTICS, INC., Portland, OR (US)

(72) Inventors: Vlad Bogin, Portland, OR (US); Thomas Ichim, San Diego, CA (US); Daqing Ma, London (GB)

(21) Appl. No.: 15/615,912

(22) Filed: Jun. 7, 2017

Related U.S. Application Data
(60) Provisional application No. 62/346,608, filed on Jun. 7, 2016.

(51) Int. Cl. A61K 33/00; A61K 9/00; A61K 9/127; A61G 10/02; A61M 5/00; A61M 16/12; A61M 37/00

(52) U.S. Cl. CPC A61K 33/00; A61M 16/12; A61K 9/0019; A61K 9/007; A61M 37/0092; A61G 10/026; A61M 5/00; A61K 9/127; A61M 2005/006; A61M 2202/0208; A61M 2202/0291

(57) ABSTRACT

Disclosed are the unique and unexpected findings that xenon gas possesses the ability to directly inhibited amyloid beta associated toxicity, inhibit production of amyloid beta, and stimulate synthesis of soluble, free-form amyloid beta. The invention teaches the use of xenon gas, as well as various combinations of Noble Gases, for the prevention, amelioration, and treatment of Alzheimer's Disease and other pathologies associated with generation of insoluble aggregates containing amyloid beta.

### CROSS-REFERENCE TO RELATED APPLICATIONS

This application claims priority to U.S. Provisional Application No. 62/346,608, filed Jun. 7, 2016, which is incorporated herein by reference in its entirety.

### FIELD OF THE INVENTION

The invention pertains to the therapeutic uses of gases, more particularly the invention pertains to the field of neuroprotection by noble gas mixtures, more particularly the invention pertains to the use of xenon and/or other noble gases for reducing neurotoxicity associated with beta amyloid peptides, as well as generation of neurotoxic beta amyloid.

### BACKGROUND OF THE INVENTION

Alzheimer's disease is a chronic neurodegenerative disorder for which there is no cure. Currently prescribed medication only temporarily relieves some of the symptoms. The main hallmarks of the disease are disorientation, loss of memory, loss of neurons and synapses in the brain, the accumulation of beta-amyloid protein in the brain (amyloid plaques), and intracellular aggregation of hyperphosphorylated tau protein (tangles). At a molecular level, amyloid plaques and neurofibrillary tangles are evident in the brains of patients with AD, marked by the presence of insoluble deposits and amyloid-beta peptide and cellular material surrounding the neurons. An essential disease process involves cleavage of the Aβ peptide from the integral membrane amyloid precursor protein (APP) by β-secretase to yield a secreted fragment of APP (sAPPβ) and a C-terminal fragment of APP (CTFβ) which undergoes further cleavage by the γ-secretase complex to yield a smaller C-terminal fragment (CTFγ) and Aβ fragments with prevalence of Aβ42 and Aβ40. Accumulation of Aβ fragments lead to their oligomerization, which mediates significant neurological damage. Accordingly, therapeutic interventions have been directed toward preventing Aβ over-production and deposition, accelerating Aβ degradation using β- and γ-secretase inhibitors/modulators, tau aggregation blockers, and immunotherapy. Although numerous agents have been utilized experimentally to block production of Aβ peptides, or to prevent their neurotoxicity, to date, none have been therapeutically useful.

The medical use of xenon and other Noble Gases is prevalent in the field of anesthesia [1-3], as well as emerging in the area of cellular protection from apoptosis or ischemia/reperfusion injury [4-7]. The utilization of xenon in the area of modulation of Aβ production, or mitigation of Aβ peptide neurotoxicity has not been contemplated previously to the current filing.

### SUMMARY OF THE INVENTION

Various aspects of the invention relating to the above are enumerated in the following paragraphs:

Aspect 1. A method of inhibiting neurotoxicity of amyloid beta on a neuronal cell, said method comprising the steps of contacting said neuronal cell with a sufficient concentration of xenon gas capable of inhibiting loss of viability in said neuronal cell.

Aspect 2. The method of aspect 1, wherein said neurotoxicity is death of a neuronal cell.

Aspect 3. The method of aspect 1, wherein said neurotoxicity is apoptosis of a neuronal cell.

Aspect 4. The method of aspect 1, wherein said neurotoxicity is autophagy of a neuronal cell.

Aspect 5. The method of aspect 1, wherein said neurotoxicity is necrosis of a neuronal cell.

Aspect 6. The method of aspect 1, wherein said neurotoxicity is hypoactivity of a neuronal cell.

Aspect 7. The method of aspect 1, wherein said neurotoxicity is hyperactivity of a neuronal cell.

Aspect 8. The method of aspect 1, wherein said amyloid beta comprises the 1-42 peptide of said amyloid beta.

Aspect 9. The method of aspect 1, wherein said neuronal cell is selected from a group of cells comprising of: a) neurons; b) microglia; c) astrocytes; and d) blood vessel associated cells.

Aspect 10. The method of aspect 1, wherein said xenon gas is delivered by inhalation.

Aspect 11. The method of aspect 1, wherein said xenon gas is delivered intranasally.

Aspect 12. The method of aspect 1, wherein said xenon gas is delivered intravenously.

Aspect 13. The method of aspect 1, wherein said xenon gas is delivered in the form of a isotonic liquid in which said xenon gas has been dissolved.

Aspect 14. The method of aspect 1, wherein said xenon gas is delivered by echogenic liposomes.

Aspect 15. The method of aspect 1, wherein said xenon gas is replaced with another Noble Gas.

Aspect 16. The method of aspect 15, wherein said another Noble Gas is helium.

Aspect 17. The method of aspect 15, wherein said another Noble Gas is neon.

Aspect 18. The method of aspect 15, wherein said another Noble Gas is krypton.

Aspect 19. The method of aspect 15, wherein said another Noble Gas is argon.

Aspect 20. The method of aspect 1, wherein said xenon is administered in a noble gas containing mixture, said mixture is comprised of a gas mixture containing oxygen and a proportion by volume of 20 to 70% of xenon.

Aspect 21. The method of aspect 20, wherein said proportion of xenon is between 22 and 60% by volume to oxygen.

Aspect 22. The method of aspect 20, wherein said proportion of xenon is between 25 and 60% by volume to oxygen.

Aspect 23. The method of aspect 17, wherein said noble gas containing mixture consists only of a) oxygen and xenon or b) air and xenon.

Aspect 24. The method of aspect 17, wherein said noble gas containing mixture also contains nitrogen, helium, Nitric Oxide, krypton, argon or neon.

Aspect 25. The method of aspect 17, wherein said noble gas containing mixture contains a proportion by volume of oxygen of between 15 and 25%.

Aspect 26. The method of aspect 17, wherein said noble gas containing mixture is supplied for inhalation from a pressurized container at a pressure greater than 2 bar.

Aspect 27. The method of aspect 17, wherein said noble gas containing mixture is administered through the use of a hyperbaric chamber.

Aspect 28. The method of aspect 25, wherein said hyperbaric chamber is pressurized to a pressure of no more than 3 atm (0.3 MPa).

Aspect 29. The method of aspect 26, wherein a noble gas is administered to the patient while the patient is in the hyperbaric environment.

Aspect 30. The method of aspect 1, wherein said xenon is used to decrease beta secretase activity in a neuronal cell population.

Aspect 31. The method of aspect 1, wherein said xenon is used to augment amount of free beta amyloid from a neuronal cell population.

### BRIEF DESCRIPTION OF THE DRAWINGS

FIG. 1A shows a graph showing cell viability challenged by Amyloid β.

FIG. 1B shows a graph showing cell viability challenged by Amyloid β with xenon.

FIG. 1C shows a graph showing cell viability challenged by Amyloid β with isoflurane-iso.

FIG. 1D shows a graph showing cell viability challenged by Amyloid β with nitrous oxide-N2O.

FIG. 2A shows a photo of an electrophoresis stain showing xenon reduces Amyloid β aggregation.

FIG. 2B shows a graph showing % of total protein versus inhalational anesthetics.

FIG. 3 shows stains comparing the effect of various inhalational anesthetics on enzymes involved in Amyloid β productions and apoptosis.

FIG. 4A shows a bar graph comparing Amyloid β in cell lysates of H4-APP cells treated with inhalational anesthetics.

FIG. 4B shows a bar graph comparing Amyloid β in culture media of H4-APP cells treated with inhalational anesthetics.

### DESCRIPTION OF THE INVENTION

The invention teaches means of utilizing xenon-containing gas mixtures to provide protection from neurotoxicity associated with amyloid beta peptides, as well as to suppress production of such peptides. The current invention provides means and methods for prophylaxis and treatment of Alzheimer's disease, and neuropathies associated with accumulation of amyloid deposits or tau mediated pathologies through administration of Nobel Gases, with preferred embodiments including administration of xenon, or xenon/argon mixtures. In one embodiment the invention provides reducing neurotoxicity of neurological disease related factors by administering xenon or xenon/argon mixtures, said factor being a form of amyloid beta (Aβ) protein, said specific form being selected from a group comprising of: a) a precursor of Aβ; b) a truncated variant of Aβ; c) a peptide derived from β; d) a pryoglutamated peptide of Aβ; e) a cross-linked beta amyloid protein species (CLAPS); f) an oxidized amyloid beta protein; g) amyloid precursor protein (APP); h) Aβ(1-42); i) Aβ(1-40); j) a peptide generated by enzymatic cleavage of APP. In another aspect, the type of Aβ which mediates neurotoxicity, which is reduced by the practice of the invention is a monomeric, a fibrillar, or a microvesicle associated form. Additionally, one of skill in the art may utilize the invention to reduce neurotoxicity of a circulating factor such as: a) tau protein, b) hyperphosphorylated tau protein; c) peptides derived from tau protein; and d) precursors to tau protein. Inhibition of said soluble, or circulating factors may be accomplished through suppression of biological (pathological) activity through binding of a ligand or activation of an inactivator factor, as well as inhibition of production of neurotoxic factors.

In one embodiment of the invention, therapeutic Noble Gas compositions are administered in a manner to alter apoptotic molecules and their ratio in the body. Specifically, the invention teaches that various concentrations of xenon gas, when delivered into circulation, either by inhalation [8-10], or administration of echogenic xenon liposomes [11, 12], can be utilized to block p53 upregulation, and/or to block the ability of p53 to induce p21 (BAX). The use of xenon has been reviewed by numerous authors in the art, which provide guidance as to details of administration [13-15]. Importantly, the new and non-obvious aspect of the current invention is that application of neuroprotective properties of xenon to selective neuroprotection of neurological tissue affected by Alzheimer's Disease.

Means of administering xenon gas therapeutically are well known in the art. Examples of clinical use of xenon are incorporated by reference to guide one of skill in the art in the practice of the invention by applying and/or modifying existing techniques of administration to the patient with Alzheimer's disease or other conditions associated with beta amyloid associated pathologies. Said examples, include; a) Sclabassi et al. [16] examined electroencephalographic changes in ten healthy adults administered xenon at concentrations of 25%, 30%, and 35% each administered for 5-minute intervals. They report no adverse events in any of the participants studied; b) Yagi et al. [17] investigated the analgesic and hypnotic effects of 21% xenon administered for 20 minutes to six healthy male volunteers. The authors reported no adverse events in any of the study participants; and c) Peterson-Felix et al. [18] administered xenon in increasing concentrations (10%, 20%, 30%, and 40%) to 12 healthy volunteers and compared the analgesic effects with equivalent concentrations of nitrous oxide. Safety was reported, which was repeated in other studies examining the use of xenon at similar or higher concentrations [16, 19]. Additionally, xenon has been used in comatose patients [8],

Xenon or a xenon-containing gas mixture are further used to produce a medicament for the treatment of amyloid associated conditions in the brain, to produce a medicament for the treatment of impairment of said neurodegenerative amyloid associated conditions and prophylaxis and/or therapy of impairments of cognitive performance. In addition, xenon or xenon-containing gas mixtures are advantageously employed as medicament for the treatment of states with amyloid associated oxygen deficiency. Xenon or a xenon-containing gas mixture are further used to produce a medicament for the treatment of cognitive or cerebral dysfunction, in particular of postoperative cognitive dysfunction after neuronal exposure to amyloid beta or peptides derived thereof. Cerebral dysfunctions caused by amyloid beta peptides relate to impairments of the microcirculation, of oxygen utilization and of metabolic functions. The medicament is thus also used to treat cerebral disorders such as impairments of the microcirculation, of oxygen utilization and of metabolic functions.

Gaseous xenon or xenon-containing gas mixtures are particularly advantageously employed for prophylaxis before establishment of amyloid plaques. Prophylactic administration of xenon or xenon-containing gas mixtures takes place for example pre-disease, during disease initiation, and disease progression.

The provided medicament for cerebral protection of amyloid associated pathologies and the indications mentioned, or the medicament produced directly on use, in particular in the direct vicinity of the patient, is for example a gas mixture which comprises from 1 to 80% by volume (based on standard conditions, i.e. 20 .degree. C., 1 bar absolute) xenon (e.g. remainder oxygen). The medicament which is administered to the patient comprises xenon in pharmacologically or therapeutically effective amount, in particular in subanesthetically or anesthetically effective amount. A medicament with xenon in subanesthetically effective amount is advantageous. Subanesthetically effective (subanesthetic) amounts of xenon mean those amounts or concentrations of xenon which are insufficient for general anesthesia. These are in general amounts of up to 70% by volume xenon, preferably up to 65% by volume, particularly preferably up to 60% by volume, in particular up to 50% by volume xenon. Pure xenon is accordingly metered into the patient's respiratory gas in the stated concentrations. This means that the respiratory gas supplied to the patient comprises for example from 5 to 60% by volume, 5 to 50% by volume, 5 to 40% by volume, 5 to 30% by volume or 5 to 20% by volume xenon. In special cases, e.g. for prophylaxis, especially during prolonged ventilation, a dosage of xenon in the respiratory gas with a low concentration, for example 1 to 35% by volume, 5 to 25% by volume or 5 to 20% by volume xenon in the respiratory gas, may be advantageous. The medicaments, in particular gaseous medicaments, preferably comprise besides xenon one or more gases or substances which are gaseous at body temperature under atmospheric pressure. Examples of gas mixtures which can be used are xenon-oxygen gas mixtures or gas mixtures of xenon and one or more inert gases such as nitrogen or a rare gas or xenon-oxygen inert gas mixtures. Admixture of a gas to the xenon may be very advantageous if it is intended to introduce little xenon into the body.

Examples of gases or gas mixtures employed as medicament for treatment of Alzheimer's Disease, or prophylaxis, or amelioration include: 1.) 100% by volume xenon; 2.) 70% by volume xenon/30% by volume oxygen; 3.) 65% by volume xenon/30% by volume oxygen/5% by volume nitrogen; 4.) 65% by volume xenon/35% by volume oxygen; 5.) 60% by volume xenon/30% by volume oxygen/10% by volume nitrogen; 6.) 60% by volume xenon/35% by volume oxygen/5% by volume nitrogen; 7.) 60% by volume xenon/40% by volume oxygen; 8.) 55% by volume xenon/25% by volume oxygen/20% by volume nitrogen; 9.) 55% by volume xenon/30% by volume oxygen/15% by volume nitrogen; 10.) 55% by volume xenon/35% by volume oxygen/10% by volume nitrogen; 11.) 55% by volume xenon/40% by volume oxygen/5% by volume nitrogen; 12.) 55% by volume xenon/45% by volume oxygen; 13.) 50% by volume xenon/50% by volume oxygen; 14.) 50% by volume xenon/45% by volume oxygen/5% by volume nitrogen; 15.) 50% by volume xenon/40% by volume oxygen/10% by volume nitrogen; 16.) 50% by volume xenon/30% by volume oxygen/20% by volume nitrogen; 17.) 50% by volume xenon/25% by volume oxygen/25% by volume nitrogen; 18.) 45% by volume xenon/55% by volume oxygen; 19.) 45% by volume xenon/50% by volume oxygen/5% by volume nitrogen; 20.) 45% by volume xenon/45% by volume oxygen/10% by volume nitrogen; 21.) 45% by volume xenon/40% by volume oxygen/15% by volume nitrogen; 22.) 45% by volume xenon/35% by volume oxygen/20% by volume nitrogen; 23.) 45% by volume xenon/30% by volume oxygen/25% by volume nitrogen; 24.) 45% by volume xenon/30% by volume oxygen/25% by volume nitrogen; 25.) 40% by volume xenon/30% by volume oxygen/30% by volume nitrogen; 26.) 40% by volume xenon/50% by volume oxygen/10% by volume nitrogen; 27.) 35% by volume xenon/25% by volume oxygen/40% by volume nitrogen; 28.) 35% by volume xenon/65% by volume oxygen; 29.) 30% by volume xenon/70% by volume oxygen; 30.) 30% by volume xenon/50% by volume oxygen/20% by volume nitrogen; 31.) 30% by volume xenon/30% by volume oxygen/40% by volume nitrogen; 32.) 20% by volume xenon/80% by volume oxygen; 33.) 20% by volume xenon/30% by volume oxygen/50% by volume nitrogen; 34.) 15% by volume xenon/30% by volume oxygen/55% by volume nitrogen; 35.) 15% by volume xenon/50% by volume oxygen/35% by volume nitrogen; 36.) 10% by volume xenon/90% by volume oxygen; 37.) 10% by volume xenon/50% by volume oxygen/40% by volume nitrogen; 38.) 10% by volume xenon/30% by volume oxygen/60% by volume nitrogen; 39.) 10% by volume xenon/25% by volume oxygen/65% by volume nitrogen; 40.) 5% by volume xenon/25% by volume oxygen/70% by volume nitrogen; 41.) 5% by volume xenon/30% by volume oxygen/65% by volume nitrogen; 42.) 5% by volume xenon/50% by volume oxygen/45% by volume nitrogen; 43.) 5% by volume xenon/30% by volume oxygen/65% by volume nitrogen; 44.) 5% by volume xenon/95% by volume oxygen; 45.) 1% by volume xenon/99% by volume oxygen; 46.) 1% by volume xenon/30% by volume oxygen/69% by volume nitrogen; 47.) 1% by volume xenon/25% by volume oxygen/74% by volume nitrogen.

In some embodiments of the invention, xenon or Noble gas containing gas mixtures are administered to enhance effects of therapeutic interventions on alzheimer's disease. Inhibition of inflammatory cytokine production by xenon of neural cells in response to amyloid beta peptides is envisioned within the scope of the invention to increase efficacy of interventions that prevent neuronal apoptosis, block glutaminergic toxicity, and enhance regenerative activity. For example, in one embodiment, patients treated with antiapoptotic agents are treated with doses of xenon sufficient to enhance said antiapoptotic effects.

In one embodiment treatment of Alzheimer's disease is performed by administration of a gas that contains between 10% and 80% by volume of xenon which is mixed together or administered to the same patient with an antioxidant. In one embodiment the antioxidant is vitamin E, a chemical analog of vitamin E or a chemical derivative of vitamin E. In another embodiment the antioxidant is Trolox, i.e. 6-hydroxy-2,5,7,8-tetramethylchromane-2-carboxylic acid, which is a water-soluble chemical analog of vitamin E. In another embodiment said xenon gas is administered to the patient before, concomitantly with or after administration of the antioxidant, preferably after administration of the antioxidant. For use in treatment the xenon is in a mixture with oxygen and/or nitrogen with the proportion of oxygen is at least 21% by volume. Furthermore the treatment gas may, in one embodiment, consist of xenon and oxygen or of xenon, nitrogen and oxygen. In one embodiment the xenon is administered to the patient by inhalation and contains a non-anesthetic amount of xenon. Furthermore, the drug mixture may consist of a gas mixture formed from oxygen and nitrogen. In one aspect of the invention the gas is administered to the patient one or more times per day with the gas administered to the patient for an inhalation time of a few minutes to a few hours, typically between 15 minutes and 6 hours, preferentially less than 4 hours. Specifically, the duration, the dosage regimen and the frequency of administration of the xenon and/or the antioxidant depend on the progression of the neurological condition of the patient under consideration and these parameters will be preferentially set by the physician or care staff depending on the neurological condition of the patient under consideration; the xenon (or the gas mixture containing the xenon) is packaged in a gas cylinder having a volume (water equivalent) ranging up to 50 liters, typically of about from 0.5 to 15 liters, and/or at a pressure of less than or equal to 350 bar absolute, typically a pressure of between 2 and 300 bar. Preferably, the gas cylinder is made of steel, aluminum or composite material, and is equipped with a valve or a pressure regulator that is integrated, making it possible to control the flow rate and optionally the pressure of the gas delivered; during the treatment, the xenon (or the gas mixture containing the xenon) is administered to the patient by inhalation by means of a face mask or nasal mask or of nasal goggles or by means of any other system for administration of an inhalable gas.

In some embodiments, the xenon composition comprises xenon gas. In some embodiments, the xenon gas is administered (e.g., by inhalation, intraocularly, or intranasally) at a concentration of 10% to 35% by volume in 21% by volume oxygen gas and a balance of nitrogen gas. In some embodiments, the xenon gas is administered at a concentration of 0.5% to 10% by volume in 21% by volume oxygen gas and a balance of nitrogen gas. In some embodiments, the argon composition comprises argon gas. In some embodiments, the argon gas is administered (e.g., by inhalation, intraocularly, or intranasally) at a concentration of 10% to 35% by volume in 21% by volume oxygen gas and a balance of nitrogen gas. In some embodiments, the argon gas is administered at a concentration of 35% to 75% by volume in 21% by volume oxygen gas and a balance of nitrogen gas. In some embodiments, the composition comprises a combination of xenon and argon gas. In some embodiments, the xenon and/or argon composition comprises a nanoparticle or nanosponge. In some embodiments, the nanoparticle or nanosponge is administered intravenously, intraarterially, intramuscularly, subcutaneously, intranasally, or intracranially in any of the method described herein, the xenon composition is administered to the subject over a continuous period of time at least one time per day. In some embodiments, the continuous period of time is at least 15 minutes. In other embodiments, the continuous period of time is at least one minute, at least two minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, or more. In some embodiments, the subject is administered the xenon and/or argon composition at least one time per day for at least one month. In some embodiments, the xenon and/or argon composition comprises xenon and/or argon gas, and can be administered, for example, at a concentration of 10% to 35% by volume in 21% by volume oxygen gas and a balance of nitrogen gas. In some embodiments, the xenon gas is administered at a concentration of 0.5% to 10% by volume in 21% by volume oxygen gas and a balance of nitrogen gas. In some embodiments, the argon gas is administered at a concentration of 35% to 75% by volume in 21% by volume oxygen gas and a balance of nitrogen gas. In one embodiment, the concentration of oxygen in the composition is not lower than 21% by volume. In some embodiments, the xenon and/or argon gas is administered by inhalation, intraocularly, or intranasally. In some embodiments, the xenon and/or argon composition comprises a nanoparticle or nanosponge, for example, for intravenous, intraarterial, intramuscular, subcutaneous, intranasal, or intracranial administration of the xenon gas. In some embodiments, the xenon and/or argon gas can be administered by self-administration. In embodiments of self-administration, the xenon and/or argon gas is typically administered intranasally or via inhalation.

The xenon and/or argon compositions can also be formulated as liquids containing xenon and/or argon gas in echogenic liposomes (e.g., 10% to 100% saturation of xenon gas, 20% to 100% saturation of xenon and/or argon gas, 40% to 100% saturation of xenon and/or argon gas, 50% to 100% saturation of xenon and/or argon gas, 60% to 100% saturation of xenon and/or argon gas, 20% to 80% saturation of xenon and/or argon gas, 40% to 80% saturation of xenon and/or argon gas, or 60% to 80% saturation of xenon and/or argon gas). A single dosage of a liquid containing liposomes can be at least 20 mL to 200 mL, or 20 mL to 100 mL. In one embodiment, 1 mL of liposomes can contain 1.8 mL xenon and/or argon gas. Liposomes that can be used in any of the methods described herein can contain a phospholipid bilayer and a core (e.g., a core of an xenon and/or argon saturated liquid or a core of xenon and/or argon gas).

The liposomes are artificial submicron vesicles that contain a phospholipid bilayer and a hydrophilic core. The phospholipid bilayer and core are ideal for incorporating a variety of gases, while maintaining the physiological inertness of its contents. Such liposomes can be used to trigger release of a gas (e.g., xenon and/or argon) from these liposomes on demand. For example, one-megahertz low-amplitude (0.18 MPa) continuous wave ultrasound can be used to release a gas (e.g., xenon and/or argon) from the liposomes, at a specific time, as they pass through the internal carotid artery, or via low intensity ultrasound focused on particular vascular beds, to specific brain areas.

Several methods that can be used to generate liposomes are known in the art. The liposomes generated can be multilamellar vesicles (MLVs), large unilamellar vesicles (LUVs), or small unilamellar vesicles (SUVs). MLVs can be generated using methods that include the addition of water to a lipid film followed by dispersal by mechanical agitation. LUVs may be generated using methods that include extrusion of preformed MLVs through filters (e.g., polycarbonate filters) with defined pore size. SUVs can be generated by sonication, French press, and homogenization procedures (Japanese Patent No. 7934-1985). Liposomes may be loaded with a therapeutic agent in one of several ways including, but not limited to, co-solubilization of the agent in an organic solvent with the lipid, and subsequently dispersing the mixture in aqueous buffer either after removing the solvent or by a reverse-phase procedure; co-dispersing the agent and the lipid in an aqueous buffer; or through the use of an active trapping procedure (e.g., where the agent is loaded after the liposomes have been formed).

In one non-limiting example, xenon and/or argon-encapsulating liposomes can be composed of 1,2-dipalmitoyl-sn-glycero-3-phosphocholine (DPPC), 1,2-dioleoyl-sn-glycero-3-phosphocholine (DOPC), and cholesterol at a molar ratio of 60:30:10. To make such liposomes, five milligrams of lipids can be mixed in chloroform, and the solvent evaporated with argon in a 50 .degree. C. water bath to form a thin film on a glass vial. The lipid film can then be placed under vacuum (<100 .mu.m Hg) for a sufficient time, e.g., 4 to 6 hours to allow complete solvent removal. The dried lipid film is then hydrated (e.g., with 0.32 mol/L mannitol) and sonicated to generate liposomes. To load the liposomes with xenon and/or argon gas, the sonicated liposomes are transferred to a 2-mL glass vial with a sealed cap, and incubated with a 10 mL mixture of xenon (70% by volume)/argon (30% by volume) mixture or vice versa, (e.g., 70% by volume argon and 30% xenon). The resulting pressurized liposomes can be stored, e.g., at −70 .degree. C. The liposomes used in the methods described herein can also contain one or more pharmaceutical agents or additional therapeutic agents for treating Alzheimer's. A single dosage of a xenon and/or argon composition (e.g., solid xenon and/or argon compositions, such as echogenic liposomes or nanosponges) can be 1 mg to 500 mg, 10 mg to 400 mg, 10 mg to 300 mg, 10 mg to 200 mg, 10 mg to 100 mg, 1 mg to 10 mg, 0.1 mg to 10 mg, and 0.1 mg to 5 mg. Nanosponges that can be used in any of the methods described herein can contain a solid external core (e.g., crosslinked cyclodextrin) that contains xenon and/or argon gas.). For example, nanosponges may be formed by crosslinked cyclodextrin with carbonyldiimidazole. In one example, the crosslinking is performed with stirring at 80-100 .degree. C. for 4 hours. The resulting solid can be washed with water and recovered by filtration. The resulting solid can be loaded with gas by exposure to a saturated solution of gas (e.g., xenon and/or argon gas).

In one embodiment of the invention xenon or other Noble Gas containing compositions are utilized together with therapeutic agents for use in the present invention induce an immune response against A.beta. peptide. These agents include A.beta. peptide itself and variants thereof, analogs and mimetics of A.beta. peptide that induce and/or crossreact with antibodies to A.beta. peptide, and antibodies or T-cells reactive with A.beta. peptide. Induction of an immune response can be active as when an immunogen is administered to induce antibodies or T-cells reactive with A.beta. in a patient, or passive, as when an antibody is administered that itself binds to A.beta. in patient. A.beta., also known as .beta.-amyloid peptide, or A4 peptide (see U.S. Pat. No. 4,666,829), is a peptide of 39-43 amino acids, which is the principal component of characteristic plaques of Alzheimer's disease. A.beta. is generated by processing of a larger protein APP by two enzymes, termed .beta. and .gamma. secretases. Known mutations in APP associated with Alzheimer's disease occur proximate to the site of .beta. or .gamma. secretase, or within A.beta.. For example, position 717 is proximate to the site of .gamma.-secretase cleavage of APP in its processing to A.beta., and positions 670/671 are proximate to the site of .beta.-secretase cleavage. It is believed that the mutations cause AD by interacting with the cleavage reactions by which A.beta. is formed so as to increase the amount of the 42/43 amino acid form of A.beta. generated. A.beta. has the unusual property that it can fix and activate both classical and alternate complement cascades. In particular, it binds to Clq and ultimately to C3bi. This association facilitates binding to macrophages leading to activation of B cells. In addition, C3bi breaks down further and then binds to CR2 on B cells in a T cell dependent manner leading to a 10,000 increase in activation of these cells. This mechanism causes A.beta. to generate an immune response in excess of that of other antigens. In one embodiment of the invention, xenon gas containing mixtures are used to reduce complement mediated inhibition of neuronal cell viability in Alzheimer's Disease.

In one embodiment the invention discloses a combination therapy using Noble Gas containing mixtures together with a therapeutically effective amount of at least one or more other pharmaceutically active drugs selected form the group consisting of: (a) drugs useful for the treatment of Alzheimer's disease, (b) drugs useful for inhibiting the deposition of amyloid protein (e.g., amyloid beta protein) in, on or around neurological tissue, (c) drugs useful for treating neurodegenerative diseases, and (d) drugs useful for inhibiting gamma-secretase, or (3) at least one pharmaceutically acceptable carrier, and an effective amount of one or more BACE inhibitors, (4) at least one pharmaceutically acceptable carrier, and effective amount of one or more cholinesterase inhibitors, or (5) at least one pharmaceutically acceptable carrier, and effective amount of one or more cholinesterase inhibitors, or (6) at least one pharmaceutically acceptable carrier, and effective amount of one or more BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA.sub.A inverse agonists; inhibitors of amyloid aggregation; glycogen synthase kinase beta inhibitors; promoters of alpha secretase activity; PDE-10 inhibitors and cholesterol absorption inhibitors, or (7) effective amount of one or more BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA.sub.A inverse agonists; inhibitors of amyloid aggregation; glycogen synthase kinase beta inhibitors; promoters of alpha secretase activity; PDE-10 inhibitors and cholesterol absorption inhibitors, or (8) at least one pharmaceutically acceptable carrier, and an effective amount of donepezil hydrochloride, or (9) an effective amount of donepezil hydrochloride.

### Examples

### Materials

Full length of amyloid-β protein (1-42) hydrochloride salt (1 mg) was purchased from Bachem Inc (Switzerland) while poly-D-lysine and MTT [3-(4,5-dimethylthiazol-2-yl)-2,5-diphenyltetrazolium bromide] was purchased from Merck Co. The stock (400 μM 1 mg in 0.55 ml of PBS) of amyloid-β1-42 was prepared and then incubated for 72 hours at 37° C. to make a toxic form of amyloid-β. The stock solutions were diluted to the desired concentrations immediately before use and added to the culture medium. Neurobasal media and B27 supplements were obtained from Invitrogen cooperation, Cytosine Arabinoside (CA), DNase (Deoxyribonuclase) and Antibiotic Antimycotic solution were purchased from sigma. Xenon, Nitrous oxide and Isoflurane were all purchased from AIR PRODUCT. Pure neuronal cortical cells were maintained routinely in Neurobasal media supplemented with B27 and AAS at 37° C. in a humidified atmosphere of 5% CO2/95% air. All cells were cultured in poly-D-lysine-coated culture dishes. The medium was changed every other day and cells were plated at an appropriate density according to each experimental scale. On day 5 after neuronal plating, 100 μl/10 ml cytosine arabinoside hydrochloride (Sigma) was added to the cell cultures to halt non-neuronal cell division. Neuronal cell cultures were ready to use on day 7. Cells were treated with 0.8 μM Amyloid-β in the presence or absence of xenon, nitrous oxide or isoflurane for 24 hrs. Anaesthetics were delivered from an Anaesthesia machine to a sealed hand-made metal chamber containing 24 well plates seeded with numerous cells. After 24 hrs, cell viability was determined by MTT assay. This study conforms to the United Kingdom Animals (Scientific Procedures) Act of 1986 and the Home Office (UK) approved the study protocol. Foetal BALB/c mice at 15-16 days gestation were gathered by Caesarean section. Whole cerebral cortices (without the basal ganglia or hippocampus) were isolated, and the meninges removed. The whole process of micro-dissection was done under the microscope with the specimens in Dissecting Solution (placed on a Petri dish containing ice) made up of Hank's Balanced Salt Solution HBSS (Gibco, UK) supplemented with anhydrous NaHCO3 (0.04M), anhydrous sucrose (0.2M) and anhydrous D-glucose (0.3M) and antibiotic-antimycotic solution (AAS, GibroBRL). The cells were then trypsinated in 0.25% trypsin for 30 minutes at 37° C. in a 120 rpm shaking chamber filled with 5% CO2. The cells were returned to the shaker for a further 30 minutes after the addition of 40 μl of DNase (Sigma, UK). The mixture was then centrifuged at 1400 rpm for 10 minutes at 4° C. and the supernatant was carefully discarded and resuspended with PM10/10 (Plating Media made up of 77% Medium Stock; 10% Foetal Bovine Serum (Gibco, UK); 10% Horse Serum (Gibco, UK); 1% Glutamine (Gibco, UK); 1% AAS (GibroBRL); 1% Murine Epidermal Growth Factor (Gibco, UK)). The mixture was then centrifuged two more times, each time discarding the supernatant and washing with PM10/10. After three cycles of centrifugation, the supernatant was discarded and replaced with 10 ml of cold Neurobasal Media (Gibco, UK) supplemented with Glutamine (25 μl/10 ml), B27 (200 μl/10 ml) (Gibco, UK) and AAS (100 μl/10 ml: final concentrations when added to the cultures: Penicillin 100 U/ml; Streptomycin Sulphate 100 μg/ml; Amphotericin B 0.25 μl/ml). Following filtration, the cells were plated at a density of 2.5×105 cells/cm2 on 24 multiwell plates coated with Poly-D-Lysine (Becton Dickinson Labware, MA) and kept in a humidified 5% CO2 incubator at 37° C. The culture medium was replaced every 24-48 hours with pre-warmed supplemented Neurobasal Media. Forty-eight hours before the amyloid β induced experiment at the fifth day after plating, 10 μl/ml of cytosine Arabinoside (CA hydrochloride, Sigma, UK) was added to the cells to halt glial proliferation and produce a pure neuronal cell culture. Only mature neuronal cultures at day 7 were used for experimentation in vitro.

3-(4,5-Dimethyl-2-thiazolyl)-2,5-diphenyl-2H-tetrazolium bromide (MTT, Merck KGaA, Germany) was diluted to a concentration of 0.5 mg/ml using Minimum Essential Medium+Earle's−L-Glutamine×1 (Gibco, UK) for use in the experiment. On completing the experiment, the media was suctioned out and 600 μl of MTT solution was pipetted into each well. The cells were then incubated for two hours. Following this, the MTT was syringed out and 1 ml of Dimethylsulfoxide (DMSO, Fisher Scientific UK Limited, Leicestershire, UK) was added to each well. Each well was then mixed with the end of the pipette to create a homogeneous purple colour. A 300 μl sample was then taken from each well and pipetted into a 96-well plate for colorimetric analysis with duplicates. Viable cells have high mitochondrial activity, and would therefore reduce the MTT compound from a yellow to a deep purple colour. The deeper colour corresponds to a higher colorimetric value, indicating higher cell viability. Results were expressed as the percentage of MTT reduction, assuming the absorbance of control cells was 100%.

For detection of monomer formation Amyloid-beta protein (1-42) hydrochloride salt (Bachem AG, Switzerland) stored at −80° C. was removed and placed on ice. 1,1,1,3,3,3-hexafluoro-2-propanol (HFIP) (Sigma-Aldrich, Poole, UK) was placed on ice in the hood and allow to cool.110 HFIP was added to 0.5 mg amyloid- [symbol] to produce a final concentration of 1 mM. The HFIP plus amyloid-beta solution was mixed on the vortex to ensure no solid remained on the vial edges. The HFIP plus amyloid-beta solution was incubated at room temperature for 60 minutes with the vial closed. The solution was then placed on ice for 10 minutes. The HFIP plus amyloid-beta solution was aliquoted into non-siliconized micro-centrifuge tubes amyloid-beta1-42). 25 ul was aliquoted per tube and the HFIP was allowed to evaporate overnight in the hood at room temperature. A thin film of 0.112 mg peptide was left at the bottom of the tube. These films were then stored until required at −80° C.

When required peptide film containing tubes were removed from the −80° C. freezer and placed on ice. 5 uL of 100% Dimethyl Sulphoxide (DMSO) solution (Sigma-Aldrich, Poole, UK) was added to the peptide to produce a 5 mM amyloid-beta solution. The solution was mixed on the vortex for 10 seconds to ensure complete resuspension of the peptide. The 5 mM peptide was then diluted with 495 uL of Minimum Essential Media (1×) liquid—without phenol red (Invitrogen Life Technologies Ltd, Paisley, UK) to produce a 50 uM solution of peptide. The protein was produced as above and was analysed immediately this was termed 0 hours. The peptide was allowed to incubate for 24 hours at 4° C., 24 hours at 37° C. and 24 hours at 4° C. plus a further 24 hour at 37° C. to determine our starting and end materials.

The peptide was produce as mentioned above and 50 ul was then aliquoted. The aliquots were fixed inside a purpose built chamber with the caps open. Xenon (50%), nitrous oxide (75%) or isoflurane (2%) in air balanced with 5% carbon dioxide (CO2) was added to the chamber until it was completely filled with the required concentration. Once filled the chamber valves were closed and the entire chamber was placed in an incubator at 37° C. for 30 minutes to allow the gases to equilibrate with the solution. The aliquot was then sealed and the chamber refilled with the aforementioned anaesthetic gases in air balanced with 5% CO2. This refill was a precautionary measure as we could not be certain the cap did not allow anaesthetic to escape from the aliquot. The entire chamber was then replaced in the incubator at 37° C. for 24 hours.

The treated samples were centrifuged at 14,000 rpm at 4° C. for 10 minutes. They were then diluted with NuPage sample buffer (Invitrogen Life Technologies Ltd, Paisley, UK) and heated for 10 minutes at 75° C. The protein within the sample was then separated by electrophoresis on a NuPage 4-12% Bis-Tris gel (Invitrogen Life Technologies Ltd, Paisley, UK). The protein was transferred to a nitrocellulose membrane and blocked for 3 hours in a solution of 2.5 g non-fat dry milk in 50 ml TBS/Tween. The membrane was then incubated with 3-amyloid antibody (concentration 1:1000) (New England Biolab, Hitchin, UK). The bands were detected with horseradish peroxidase-linked Ig anti-rabbit antibody (concentration 1:1000) (New England Biolab, Hitchin, UK) and visualised with the enhanced chemiluminesence system (ECL, Amersham Biosciences, Little Chalfont, UK). The monomer, LMW and HMW bands were located using a protein ladder to determine the molecular mass and were then quantified by densitometry (Adobe Photoshop).

The densitometry results were then normalised to the total protein concentration within the supernatant as was determined by the centrifugation analysis.

Naïve H4 human neuroglioma cells (H4-naïve) and H4 human neuroglioma cells stably transfected to express full length human APP (H4-APP) were used. H4-naïve cells are used as a control, whilst H4 APP cells are used as an example of AD aetiology.

Cells are cultured in a medium made of 500 ml DMEM (Dulbecco's Modified Eagle Medium) (Sigma, USA), 5 ml L-Glutamine (Sigma, USA), 5 ml Penicillin-streptomycin and 100 ml Fetal bovine serum (FBS) (HyClone Laboratories). In addition to this, H4-APP cells required 2.2 ml of G418 Disulphate salt solution (Sigma, USA) H4-APP cells were transfected with a plasmid including the gene for human APP and antibiotic resistance to G418 enable selection.

H4-naïve and H4-APP cells were plated and stored in a humidified incubator with 5% CO2 at 37° C. until confluent, with the cell media being changed every other day. Once achieved, cells seeded at a density of 7×105 per well in a 6 well plate and the remaining cells were split into new flasks or stored in liquid nitrogen. Cells in the 6 well plates were allowed to acclimatise overnight and gas exposure experiments were conducted on the following day. Immediately before an experiment, cell media was changed to warm serum free media (5 ml L-glutamine, 5 ml Penicillin-Streptomycin in 500 ml DMEM, plus 2.2 ml G418 for H4-APP cells). The table below illustrates the conditions for gas exposure experiments that were conducted. In all experiments 21% 02 and 5% CO2 was used. The xenon, nitrous oxide and isoflurane experiments were conducted in an air-tight, temperature controlled chamber. The chamber was prefilled with the specific concentration of gases and cells are stored here for 6 hours. Control conditions expose H4 naïve cells to 21% 02 and 5% CO2 for 6 hours. After completion of gas exposure, protein samples were collected from cell lysates on ice in order to maintain protein viability. Culture media were also harvested to measure soluble release from cells. Enzyme-linked immunosorbent assay is a method used to determine Aβ in cell lysates and culture medium.

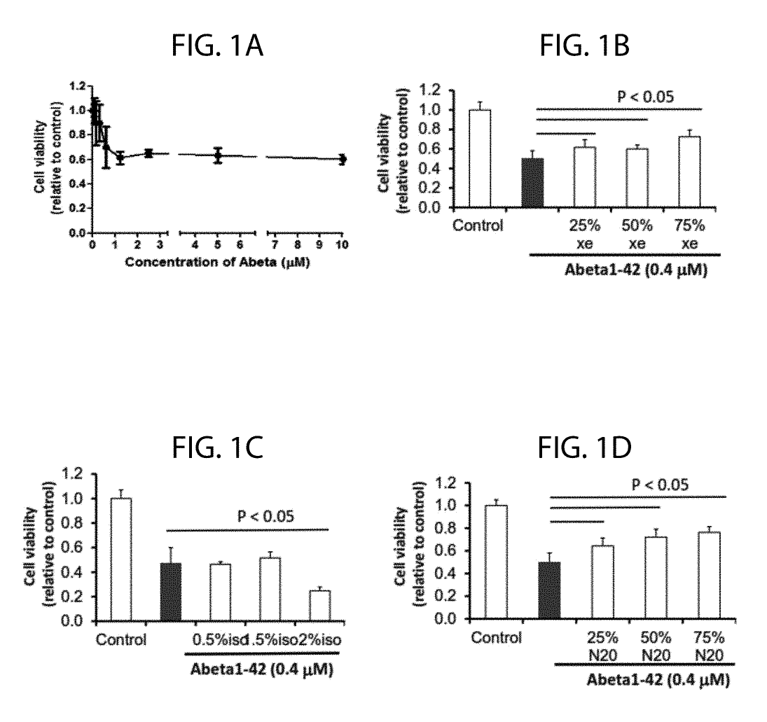

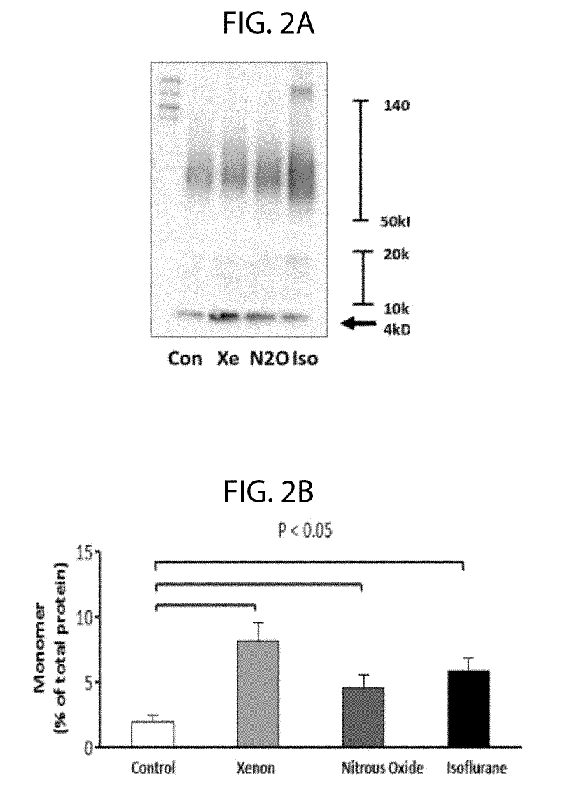

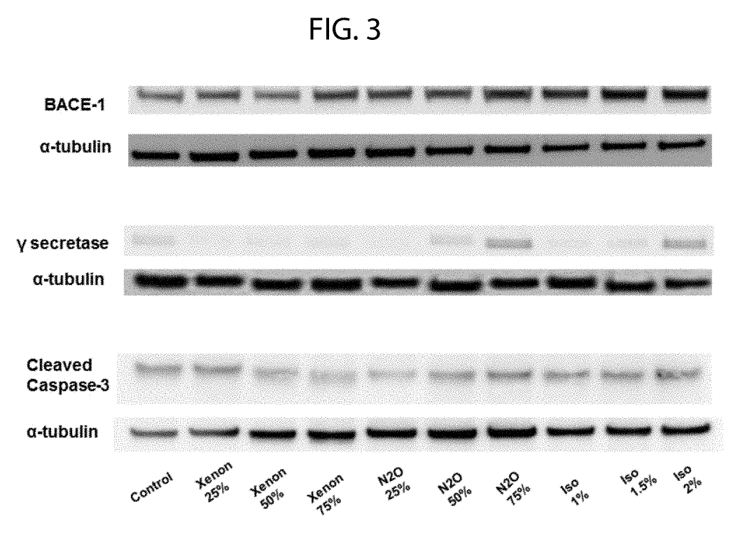

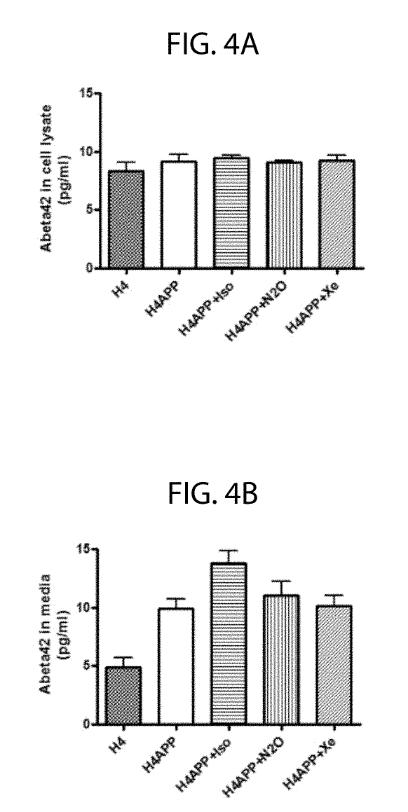

Results are shown in FIGS. 1-4.

### REFERENCES

1. Rossaint, R., et al., Multicenter randomized comparison of the efficacy and safety of xenon and isoflurane in patients undergoing elective surgery. Anesthesiology, 2003. 98(1): p. 6-13.
2. Baumert, J. H., et al., Xenon or propofol anaesthesia for patients at cardiovascular risk in non-cardiac surgery. Br J Anaesth, 2008. 100(5): p. 605-11.
3. Coburn, M., et al., Randomized controlled trial of the haemodynamic and recovery effects of xenon or propofol anaesthesia. Br J Anaesth, 2005. 94(2): p. 198-202.
4. Coburn, M., M. Maze, and N. P. Franks, The neuroprotective effects of xenon and helium in an in vitro model of traumatic brain injury. Crit Care Med, 2008. 36(2): p. 588-95.
5. Khajuria, A., et al., Anesthetics attenuate ischemia-reperfusion induced renal injury: effects and mechanisms. Acta Anaesthesiol Taiwan, 2014. 52(4): p. 176-84.
6. Wu, L., et al., Cellular signaling pathways and molecular mechanisms involving inhalational anesthetics-induced organoprotection. J Anesth, 2014. 28(5): p. 740-58.
7. Zhao, H., et al., Early treatment with xenon protects against the cold ischemia associated with chronic allograft nephropathy in rats. Kidney Int, 2014. 85(1): p. 112-23.
8. Laitio, R., et al., Effect of Inhaled Xenon on Cerebral White Matter Damage in Comatose Survivors of Out-of-Hospital Cardiac Arrest: A Randomized Clinical Trial. JAMA, 2016. 315(11): p. 1120-8.
9. Arola, O. J., et al., Feasibility and cardiac safety of inhaled xenon in combination with therapeutic hypothermia following out-of-hospital cardiac arrest. Crit Care Med, 2013. 41(9): p. 2116-24.
10. Azzopardi, D., et al., Moderate hypothermia within 6 h of birth plus inhaled xenon versus moderate hypothermia alone after birth asphyxia (TOBY-Xe): a proof-of-concept, open-label, randomised controlled trial. Lancet Neurol, 2015.
11. Britton, G. L., et al., In vivo therapeutic gas delivery for neuroprotection with echogenic liposomes. Circulation, 2010. 122(16): p. 1578-87.
12. Peng, T., et al., Therapeutic time window and dose dependence of xenon delivered via echogenic liposomes for neuroprotection in stroke. CNS Neurosci Ther, 2013. 19(10): p. 773-84.
13. Maze, M., Preclinical neuroprotective actions of xenon and possible implications for human therapeutics: a narrative review. Can J Anaesth, 2016. 63(2): p. 212-26.
14. Sanders, R. D. and M. Maze, Xenon: from stranger to guardian. Curr Opin Anaesthesiol, 2005. 18(4): p. 405-11.
15. Esencan, E., et al., XENON in medical area: emphasis on neuroprotection in hypoxia and anesthesia. Med Gas Res, 2013. 3(1): p. 4.
16. Sclabassi, R. J., et al., Effect of low concentration stable xenon on the EEG power spectrum. Electroencephalogr Clin Neurophysiol, 1987. 67(4): p. 340-7.
17. Yagi, M., et al., Analgesic and hypnotic effects of subanaesthetic concentrations of xenon in human volunteers: comparison with nitrous oxide. Br J Anaesth, 1995. 74(6): p. 670-3.
18. Petersen-Felix, S., et al., Comparison of the analgesic potency of xenon and nitrous oxide in humans evaluated by experimental pain. Br J Anaesth, 1998. 81(5): p. 742-7.
19. Lorenz, M., et al., Effects of 33% stable xenon/O2 mixture on somatosensory evoked potentials. Neurol Res, 1991. 13(2): p. 133-5.

1. A method of inhibiting neurotoxicity of amyloid beta on a neuronal cell, said method comprising the steps of contacting said neuronal cell with a sufficient concentration of xenon gas capable of inhibiting loss of viability in said neuronal cell.

2. The method of claim 1, wherein said neuronal cell is selected from a group of cells comprising of: a) neurons; b) microglia; c) astrocytes; and d) blood vessel associated cells.

3. The method of claim 1, wherein said xenon gas is delivered by inhalation

4. The method of claim 1, wherein said xenon gas is delivered intravenously.

5. The method of claim 1, wherein said xenon gas is delivered by echogenic liposomes.

6. The method of claim 1, wherein said xenon gas is replaced or used in combination with another Noble Gas.

7. The method of claim 6, wherein said another Noble Gas is selected from a group comprising of: a) helium; b) neon; c) krypton; and d) argon.

8. The method of claim 1, wherein said xenon is administered in a noble gas containing mixture, said mixture is comprised of a gas mixture containing oxygen and a proportion by volume of 20 to 70% of xenon.

9. The method of claim 8, wherein said proportion of xenon is between 22 and 60% by volume to oxygen.

10. The method of claim 9, wherein said proportion of xenon is between 25 and 60% by volume to oxygen.

11. The method of claim 1, wherein said noble gas containing mixture contains a proportion by volume of oxygen of between 15 and 25%.

12. The method of claim 1, wherein said noble gas containing mixture is supplied for inhalation from a pressurized container at a pressure greater than 2 bar.

13. The method of claim 1, wherein said noble gas containing mixture is administered through the use of a hyperbaric chamber.

14. The method of claim 13, wherein said hyperbaric chamber is pressurized to a pressure of no more than 3 atm (0.3 MPa).

15. The method of claim 13, wherein a noble gas is administered to the patient while the patient is in the hyperbaric environment.

16. The method of claim 1, wherein said xenon is used to decrease beta secretase activity in a neuronal cell population.

17. The method of claim 1, wherein said xenon is used to augment amount of free beta amyloid from a neuronal cell population.
